(19) United States
(12) Patent Application Publication
(10) Pub. No.: US 2007/0123511 A1
(43) Pub. Date: May 31, 2007

(54) Benzopyranone compounds, compositions thereof, and methods for treating or preventing cancer

(76) Inventors: Steven T. Sakata, San Diego, CA (US); Rama Krishna Narla, San Diego, CA (US); Bernd Stein, San Diego, CA (US); Martin Missbach, Gipf-Oberfrick (CH); Johanne Renaud, Basel (CH)

Correspondence Address: JONES DAY, 222 EAST 41ST ST, NEW YORK, NY, 10017, US

(21) Appl. No.: 11/473,564

(22) Filed: Jun. 23, 2006

Related U.S. Application Data
(60) Provisional application No. 60/693,867, filed on Jun. 24, 2005.

(51) Int. Cl. A61K 31/55; C07D 405/02

(52) U.S. Cl. 514/217.03; 540/596

(57) ABSTRACT

This invention relates to Benzopyranone Compounds, compositions comprising a Benzopyranone Compound and methods for treating or preventing cancer or inhibiting the growth of a cancer cell or neoplastic cell comprising administering an effective amount of a Benzopyranone Compound to a patient in need thereof. The Benzopyranone Compounds have the formula: 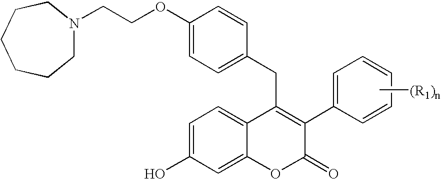
including pharmaceutically acceptable salts thereof, wherein R₁ and n are as defined herein.

This application claims the benefit of U.S. provisional application No. 60/693,867, filed June 24, 2005, the contents of which are incorporated by reference herein in their entirety.

### 1. FIELD OF THE INVENTION

This invention relates to Benzopyranone Compounds, compositions comprising a Benzopyranone Compound, and methods for using a Benzopyranone Compound to treat or prevent cancer.

### 2. BACKGROUND OF THE INVENTION

### 2.1 Cancer

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multi-step process that begins with minor preneoplastic changes, which can under certain conditions progress to neoplasia. The neoplastic lesion can evolve clonally and develop an increasing capacity for invasion, growth, metastasis, and heterogeneity, especially under conditions in which the neoplastic cells escape the host's immune surveillance. Roitt, I., Brostoff, J and Kale, D., Immunology, 17.1-17.12 (3rd ed., Mosby, St. Louis, Mo., 1993).

Descriptions of only a few types of cancers are provided below.

Characteristics of other types of cancers are well known to medical practitioners, and are described in the medical literature.

### 2.2 Brain Cancer and Brain Metastasis

There are about 10,000 incidences of brain tumors each year, and about 4000 incidences of spinal cord tumors each year (Komblith et al.(1985), Cancer: Principles and Practice of Oncology, 2^{nd} Ed., DeVita, V., Hellman, S., Rosenberg, S., eds., J. B. Lippincott Company, Philadelphia, Chapter 41: Neoplasms of the Central Nervous System). Central nervous system (CNS) tumors comprise the most common group of solid tumors in young patients (Id). Gliomas comprise about 60% of all primary CNS tumors, with the most common cerebral primary tumors being astrocytomas, meningioma, oligodendroglioma and histocytic lymphoma (Id). Gliomas usually occur in the cerebral hemispheres of the brain, but can be found in other areas such as the optic nerve, brain stem or cerebellum (Brain Tumor Society; www/tbts.org/primary.htm).

Gliomas are classified into groups according to the type of glial cell from which they originate (Id). The most common types of glioma are astrocytomas. These tumors develop from star-shaped glial cells called astrocytes. Astrocytomas are assigned to grades according to their malignancy. Low-grade astrocytomas, also known as grade I and II astrocytomas, are the least malignant, grow relatively slow and can often be completely removed using surgery. Mid-grade astrocytomas, also known as grade III astrocytomas, grow more rapidly and are more malignant. Grade III astrocytomas are treated with surgery followed by radiation and some chemotherapy. High-grade astrocytomas, also known as grade IV astrocytomas, grow rapidly, invade nearby tissue, and are very malignant. Grade IV astrocytomas are usually treated with surgery followed by a combination of radiation therapy and chemotherapy. Glioblastoma multiforme are grade IV astrocytomas, which are among the most malignant and deadly primary brain tumors (Id).

Traditionally, treatment of astrocytomas has involved surgery to remove the tumor, followed by radiation therapy. Chemotherapy can also be administered either before or after radiation therapy (Komblith et al. (1985), Cancer: Principles and Practice of Oncology, 2^{nd} Ed., DeVita, V., Hellman, S., Rosenberg, S., eds., J. B. Lippincott Company, Philadelphia, Chapter 41: Neoplasms of the Central Nervous System). While the same surgical techniques and principles have applied to treating glioblastoma multiforme and less malignant brain tumors, total removal of a glioblastoma multiforme tumor has been more difficult to achieve (Id).

The prognosis for a patient diagnosed as having a grade IV astrocytoma brain tumor has traditionally been poor. While a person treated for a grade I astrocytoma can commonly survive 10 years or more without recurrence, the mean length of survival for a patient with a grade IV astrocytoma tumor is 15 weeks after surgical treatment. Because of the high malignant-growth potential of grade IV astrocytoma tumors, only 5% of patients have survived for 1 year following surgical treatment alone, with a near 0% survival rate after 2 years. Radiation treatment in combination with surgical treatment increases the survival rate to about 10% after 2 years of treatment; however, virtually no patients survive longer than 5 years (Id).

### 2.3 Current Cancer Therapy

Currently, cancer therapy involves surgery, chemotherapy and/or radiation treatment to eradicate neoplastic cells in a patient (see, for example, Stockdale, 1998, “Principles of Cancer Patient Management”, in Scientific American: Medicine, vol. 3, Rubenstein and Federman, eds., Chapter 12, Section IV). All of these approaches pose significant drawbacks for the patient. Surgery, for example, can be contraindicated due to the health of the patient or may be unacceptable to the patient. Additionally, surgery may not completely remove the neoplastic tissue. Radiation therapy is effective only when the irradiated neoplastic tissue exhibits a higher sensitivity to radiation than normal tissue, and radiation therapy can also often elicit serious side effects. (Id.) With respect to chemotherapy, there are a variety of chemotherapeutic agents available for treatment of neoplastic disease. However, despite the availability of a variety of chemotherapeutic agents, chemotherapy has many drawbacks (see, for example, Stockdale, 1998, “Principles Of Cancer Patient Management” in Scientific American Medicine, vol. 3, Rubenstein and Federman, eds., ch. 12, sect. 10). Almost all chemotherapeutic agents are toxic, and chemotherapy causes significant, and often dangerous, side effects, including severe nausea, bone marrow depression, immunosuppression, etc. Additionally, many tumor cells are resistant or develop resistance to chemotherapeutic agents through multi-drug resistance.

Nitrosourea chemotherapeutic agents have normally been used in the treatment of brain tumors. The key property of these compounds is their ability to cross the blood-brain barrier. 1-3-bis-2-chloroethyl-1-nitrosourea (BCNU, also known as Carmustine) was the first of these to be used clinically. While the use of BCNU in combination with surgery and/or radiation treatment has been shown to be beneficial, it has not cured glioblastoma multiforme brain tumors. Additionally, complications with prolonged nitrosourea treatment have been reported (Cohen et al., Cancer Treat. Rep. 60, 1257-1261 (1976)). These complications include pulmonary fibrosis, hepatic toxicity, renal failure and cases of secondary tumors associated with nitrosourea treatment.

The use of estrogen receptor modulators Tamoxifen and Raloxifene in cancer treatment has also been investigated. Tamoxifen has been used in human clinical trials involving the treatment of recurrent malignant glial tumors (Couldwell et al., Clin. Cancer Res. 2, 619-622 (1996)). Raloxifene has been shown to inhibit metastasis of a tail tumor to the lungs in a rat model (Neubauer et al., Prostate 27, 220-229 (1995)).

While a treatment regimen of surgery, radiation therapy and chemotherapy offers the opportunity for a modestly increased lifespan for patients with a grade IV astrocytoma brain tumor, the risks associated with each method of treatment are many. The benefits of treatment are minimal, and treatment can significantly decrease the quality of the patient's brief remaining lifespan.

Accordingly, there remains a clear need in the art for anti-cancer compounds and treatment methods that overcome the disadvantages of the above-mentioned traditional approaches.

Citation or identification of any reference in Section 2 of this application is not an admission that the reference is prior art to the present application.

### 3. SUMMARY OF THE INVENTION

The present invention relates to compounds of formula (I) and (II): 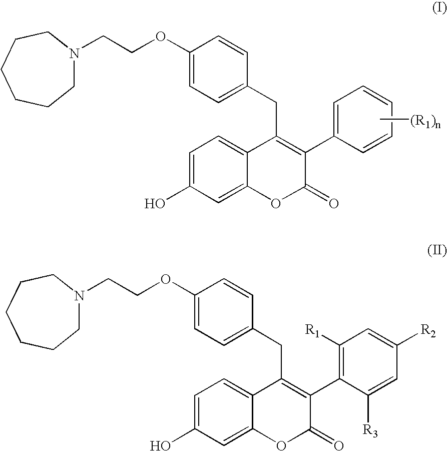
and pharmaceutically acceptable salts, hydrates, solvates, clathrates, polymorphs, prodrugs and stereoisomers thereof, wherein R₁, R₂, R₃ and n are as defined below.

A compound of formula (I), formula (II), and pharmaceutically acceptable salts, hydrates, solvates, clathrates, polymorphs, prodrugs and stereoisomers thereof (each being a “Benzopyranone Compound”), are useful for treating or preventing cancer in a patient.

The invention also relates to compositions comprising an effective amount of a Benzopyranone Compound and a pharmaceutically acceptable carrier or vehicle. The compositions are useful for treating or preventing cancer in a patient.

The invention further relates to single unit dosage forms comprising an effective amount of a Benzopyranone Compound and a pharmaceutically acceptable carrier or vehicle. The single unit dosage forms are useful for treating or preventing cancer in a patient.

The invention further relates to methods for treating or preventing cancer, comprising administering to a patient in need thereof an effective amount of a Benzopyranone Compound, or composition thereof.

The invention still further relates to methods for inhibiting the growth of a cancer cell or neoplastic cell, comprising contacting the cell with an effective amount of a Benzopyranone Compound, or composition thereof.

The invention still further relates to methods for inhibiting the growth of a tumor, for example a solid tumor, comprising contacting the tumor with an effective amount of a Benzopyranone Compound, or composition thereof.

### 4. DETAILED DESCRIPTION OF THE INVENTION

### 4.1 Definitions

As used herein, the term “halogen” means fluoro, chloro, bromo or iodo.

As used herein, the term “trifluoromethyl” means —CF₃.

As used herein, the terms “prevent”, “preventing” and “prevention” include a reduction of the risk of acquiring a given disease or disorder (e.g., cancer). In one embodiment, the Benzopyranone Compounds are administered as a preventative measure to a patient, preferably a human, having a genetic predisposition to a disease or disorder (e.g., cancer) or to a patient, preferably a human, who is at risk for a given disease or disorder (e.g., cancer) due to environmental factors (e.g., from smoking or exposure to one or more certain carcinogenic agents).

As used herein, the terms “treat”, “treating” and “treatment” include the eradication, removal, modification, or control of a tumor or primary, regional, or metastatic cancer cells or tissue and the minimization or delay of the spread of cancer.

As used herein, the term “patient” means an animal, including, but not limited, to an animal such as a human, monkey, cow, horse, sheep, pig, chicken, turkey, quail, cat, dog, mouse, rat, rabbit or guinea pig, in one embodiment a mammal and in another embodiment a human. In certain embodiments, the patient can be an infant, adolescent or adult. In a particular embodiment, the patient has or is susceptible to having (e.g., through genetic or environmental factors) cancer. In a further embodiment, the patient has or is susceptible to having (e.g., through genetic or environmental factors) a tumor.

As used herein, the term “effective amount” when used in connection with a Benzopyranone Compound means an amount of the Benzopyranone Compound effective for treating or preventing a disease or disorder disclosed herein, in one embodiment cancer.

The phrase “pharmaceutically acceptable salt,” as used herein includes, but is not limited to, salts of acidic or basic groups of a compound of formula (I) or (II). Compounds included in the present methods and compositions that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. In a preferred embodiment, the compound is in the form of a hydrochloride salt. Compounds included in the present methods and compositions that include an amino moiety can form pharmaceutically or cosmetically acceptable salts with various amino acids, in addition to the acids mentioned above. Compounds included in the present methods and compositions that are acidic in nature are capable of forming base salts with various pharmacologically or cosmetically acceptable cations. Examples of such salts include alkali metal or alkaline earth metal salts and, particularly, calcium, magnesium, sodium lithium, zinc, potassium, and iron salts.

As used herein, the term “polymorph” refers to solid crystalline forms of a Benzopyranone Compound or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a Benzopyranone Compound or a salt thereof, that further includes a stoichiometric or non-stoichiometric amount of water bound by non-covalent intermolecular forces.

As used herein, he term “clathrate” means a Benzopyranone Compound or a salt thereof in the form of a crystal lattice that contains spaces (e.g., channels) that have a guest molecule (e.g., a solvent or water) trapped within.

As used herein and unless otherwise indicated, the term “prodrug” means a Benzopyranone Compound derivative that can hydrolyze, oxidize, or otherwise react under biological conditions (in vitro or in vivo) to provide an active compound, particularly a Benzopyranone Compound. Examples of prodrugs include, but are not limited to, derivatives and metabolites of a Benzopyranone Compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “single unit dosage form” includes tablets; caplets; capsules, such as soft elastic gelatin capsules; cachets; sachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient. Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient.

As used herein and unless otherwise indicated, the term “stereoisomer” means one stereoisomer of a Benzopyranone Compound is substantially free of other stereoisomers of that Benzopyranone Compound. For example, a stereomerically pure compound having one chiral center will be substantially free of the opposite enantiomer of the compound. A stereomerically pure a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

The present invention can be understood more fully by reference to the detailed description and examples, which are intended to exemplify non-limiting embodiments of the invention.

### 4.2 The Benzopyranone Compounds

The present invention relates to compounds of formula (I): 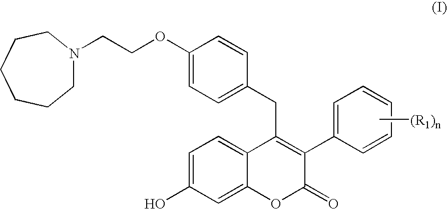
and pharmaceutically acceptable salts, hydrates, solvates, clathrates, polymorphs, prodrugs and stereoisomers thereof, wherein:

R₁ is at each occurrence independently halogen or trifluoromethyl; and

n is 1, 2or 3.

In one embodiment, n is 1.

In another embodiment, n is 2.

In another embodiment, n is 3.

In another embodiment, R₁ is halogen.

In another embodiment, R₁ is fluoro.

In another embodiment, R₁ is chloro.

In another embodiment, R₁ is trifluoromethyl.

In another embodiment, n is 1 and R₁ is halogen (e.g., fluoro or chloro).

In another embodiment, n is 1 and R₁ is trifluoromethyl.

In another embodiment, n is 2 and R₁ is halogen (e.g., chloro or fluoro).

In another embodiment, n is 2 and one R₁ group is halogen (e.g., chloro or fluoro) and the other R₁ group is trifluoromethyl.

In another embodiment, n is 3 and two R₁ groups are halogen (e.g., chloro or fluoro) and the other R₁ group is trifluoromethyl.

In another embodiment, the compounds of formula (I) do not include 4-(4-(2-(azepan-1-yl)ethoxy)benzyl)-3-(2-chloro-5-(trifluoromethyl)phenyl)-7-hydroxy-2H-chromen-2-one, 4-(4-(2-(azepan-1-yl)ethoxy)benzyl)-3-(2,6-dichloro-4-(trifluoromethyl)phenyl)-7-hydroxy-2H-chromen-2-one, 4-(4-(2-(azepan-1-yl)ethoxy)benzyl)-3-(2-chloro-4-(trifluoromethyl)phenyl)-7-hydroxy-2H-chromen-2-one, or 4-(4-(2-(azepan-1-yl)ethoxy)benzyl)-3-(4-chloro-2-(trifluoromethyl)phenyl)-7-hydroxy-2H-chromen-2-one.

The present invention further relates to compounds of formula (II): 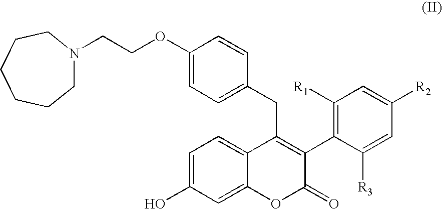
and pharmaceutically acceptable salts, hydrates, solvates, clathrates, polymorphs, prodrugs and stereoisomers thereof, wherein:

R₁, R₂ and R₃ are at each occurrence independently H, halogen or trifluoromethyl, wherein at least one of R₁, R₂ and R₃ is halogen or trifluoromethyl.

In another embodiment, one of R₁, R₂ and R₃ is halogen.

In another embodiment, one of R₁, R₂ and R₃ is fluoro.

In another embodiment, one of R₁, R₂ and R₃ is chloro.

In another embodiment, one of R₁, R₂ and R₃ is trifluoromethyl.

In another embodiment, two of R₁, R₂ and R₃ are halogen.

In another embodiment, two of R₁, R₂ and R₃ are fluoro.

In another embodiment, two of R₁, R₂ and R₃ are chloro.

In another embodiment, two of R₁, R₂ and R₃ are trifluoromethyl.

In another embodiment, R₁, R₂ and R₃ are halogen.

In another embodiment, R₁, R₂ and R₃ are fluoro.

In another embodiment, R₁, R₂ and R₃ are chloro.

In another embodiment, R₁, R₂ and R₃ are trifluoromethyl.

In another embodiment, one of R₁, R₂ and R₃ is halogen and the others are H.

In another embodiment, one of R₁, R₂ and R₃ is trifluoromethyl and the others are H.

In another embodiment, two of R₁, R₂ and R₃ are halogen and the other is H.

In another embodiment, two of R₁, R₂ and R₃ are halogen and the other is trifluoromethyl.

In another embodiment, one of R₁, R₂ and R₃ is halogen, one of R₁, R₂ and R₃ is trifluoromethyl and the other is H.

In another embodiment, R₂ is halogen or CF₃ and R₁ and R₃ are H.

In another embodiment R₁ and R₂ are halogen and R₃ is H.

Illustrative Benzopyranone Compounds are shown below in Table 1:

TABLE 1

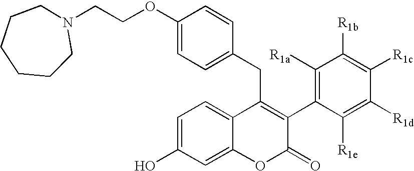

| Compound | R₁ₐ | R_{1b} | R_{1c} | R_{1d} | R₁ₑ |
|---|---|---|---|---|---|
| 1 | Cl | H | Cl | H | H |
| 2 | H | H | F | H | H |
| 3 | H | H | CF₃ | H | H |
| 4 | Cl | H | H | CF₃ | H |
| 5 | Cl | H | CF₃ | H | Cl |
| 6 | Cl | H | CF₃ | H | H |
| 7 | CF₃ | H | Cl | H | H |

### 4.3 Methods for Obtaining the Benzopyranone Compounds

The Benzopyranone Compounds can be made by one skilled in the art using known techniques, as well as by the synthetic routes disclosed herein. For example, the Benzopyranone Compounds of formula (I) can be synthesized by general Reaction Scheme 1, below.

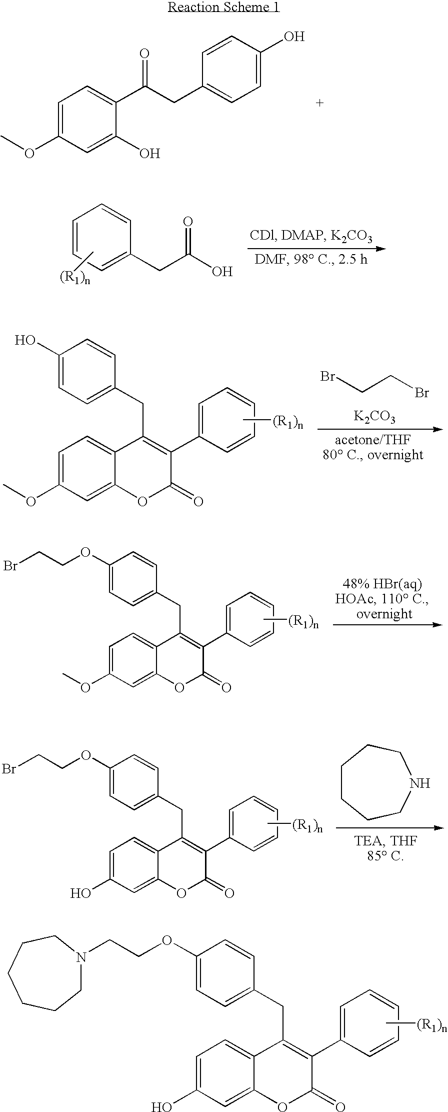

Benzopyranone Compounds of formula (II) can be prepared according Reaction Scheme 1 using the following as the carboxylic acid starting material:

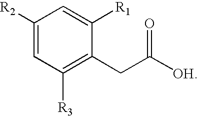

Some of the Benzopyranone Compounds can form solvates with water or other organic solvents. Such solvates are similarly included within the scope of this invention.

### 4.4 Therapeutic and Prophylactic uses of the Benzopyranone Compounds

Due to their activity, the Benzopyranone Compounds are advantageously useful in veterinary and human medicine. In particular, the Benzopyranone Compounds are useful for the treatment or prevention of cancer.

Accordingly, the present invention provides methods for the treatment or prevention of cancer comprising administering an effective amount of a Benzopyranone Compound to a patient in need thereof. In a preferred embodiment, the patient is a human. In another preferred embodiment, the Benzopyranone Compound is administered orally.

In one embodiment, the cancer is of the head, neck, eye, mouth, throat, esophagus, chest, bone, lung, colon, rectum, stomach, prostate, breast, ovaries, uterus, testicles (or other reproductive organs), skin, thyroid, blood, lymph nodes, kidney, liver, pancreas, brain or central nervous system.

In another embodiment, the cancer has metastasized. In certain embodiments, the metastasized cancer originated in the lung (both small cell or non-small cell), breast, from an unknown primary tumor, a melanoma or colon.

In another embodiment, the cancer is a primary brain cancer.

In certain embodiments, the cancer to be treated or prevented in the present invention includes, but is not limited to, a primary intracranial central nervous system tumor. Primary intracranial central nervous system tumors include glioblastoma multiforme; malignant astrocytomas; oligdendroglioma; ependymoma; low-grade astrocytomas; meningioma; mesenchymal tumors; pituitary tumors; nerve sheath tumors such as schwannomas; central nervous system lymphoma; medulloblastoma; primitive neuroectodermal tumors; neuron and neuron/glial tumors; craniopharyngioma; germ cell tumors; and choroid plexus tumors.

In other embodiments, the cancer to be treated or prevented in the present invention includes, but is not limited to, a primary spinal tumor such as a schwannoma, meningioma, ependymoma, sarcoma, astrocytoma, glioma, vascular tumor, chordoma and epidermoid.

In other embodiments, the cancer to be treated or prevented in the present invention includes, but is not limited to, a primary tumor responsible for brain metastasis such as lung (both small cell and non-small cell), breast, unknown primary, melanoma and colon.

In other embodiments, the cancer to be treated or prevented in the present invention includes, but is not limited to, a solid tumor such as a solid tumor of the breast, colon, prostate, pancreas, ovaries or uterus. In another embodiment, the solid tumor is a glioma or non-small cell lung cancer.

In another embodiment, the cancer is leukemia.

In another embodiments, the cancer is a multi-drug resistant cancer (e.g., uterine cancer).

### 4.5 Compositions Comprising a Benzopyranone Compound

When administered to a patient, e.g., an animal for veterinary use or to a human for clinical use, the Benzopyranone Compounds can be in isolated form. By “isolated” it is meant that prior to administration, a Benzopyranone Compound is separated from other components of a synthetic organic chemical reaction mixture or natural product source, e.g., plant matter, tissue culture, bacterial broth, solvent, reactants, other products, and the like. In one embodiment, the Benzopyranone Compounds are isolated via conventional techniques, e.g., extraction followed by chromotography, recrystalization, precipitation by addition of an anti-solvent or another conventional technique. When in isolated form, the Benzopyranone Compounds are at least 90%, at least 95%, at least 98%, at least 99% or at least 99.9% of a single Benzopyranone Compound by weight of that which is isolated. “Single Benzopyranone Compound” means an enantiomer or a racemate of a Benzopyranone Compound.

The Benzopyranone Compounds are advantageously administered in the form of a composition, in one embodiment a pharmaceutical composition. These compositions can be administered by any convenient route, for example by infusion or bolus injection, by absorption through epithelial or mucocutaneous linings (e.g., oral mucosa, rectal and intestinal mucosa) or via a convection-enhanced drug delivery system and can be administered together with another active agent. Administration can be systemic or local. Various delivery systems are known, e.g., encapsulation in liposomes, microparticles, microcapsules, capsules, and can be used to administer a Benzopyranone Compound of the invention. In certain embodiments, more than one Benzopyranone Compound of the invention is administered to a patient. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically to the ears, nose, eyes, or skin. In a preferred embodiment, administration is oral. A particular mode of administration can be left to the discretion of the practitioner, and can depend in-part upon the particular site of the cancer.

In one embodiment, the Benzopyranone Compound is administered in combination with another therapeutic agent or prophylactic agent. In a certain embodiment, the therapeutic agent or prophylactic agent is a chemotherapeutic agent.

In another embodiment, a Benzopyranone Compound is administered locally to the area in need of treatment. This can be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of the primary brain cancer or brain metastasis.

In certain embodiments, a Benzopyranone Compound is administered into the central nervous system by any suitable route, including intraventricular and intrathecal injection. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the Benzopyranone Compounds can be formulated as a suppository, with traditional binders and carriers such as triglycerides.

In one embodiment, a Benzopyranone Compound is administered via a convection-enhanced drug delivery system. In another embodiment, the Benzopyranone Compound is administered via a convection-enhanced drug delivery system such as that described in U.S Pat. No. 5,720,720, incorporated by reference herein. Convection-enhanced drug delivery involves positioning the tip of an infusion catheter within a tissue (e.g., brain tissue) and supplying the drug (e.g., a Benzopyranone Compound) through the catheter while maintaining a positive pressure gradient from the tip of the catheter during infusion. The catheter is connected to a pump which delivers the drug and maintains the desired pressure gradient throughout delivery of the drug. Drug delivery rates are typically about 0.5 to about 4.0 ml/min with infusion distances of about 1 cm or more. This method is particularly useful for the delivery of drugs to the brain and other tissue, particularly solid nervous tissue. In certain embodiments, convection-enhanced drug delivery is useful for delivering a Benzopyranone Compound in combination with a high molecular-weight polar molecule such as growth factors, enzymes, antibodies, protein conjugates and genetic vectors to the brain or other tissue. In these embodiments, inflow rates can be up to about 15.0 ml/min.

In another embodiment, the Benzopyranone Compounds of the invention can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, a Benzopyranone Compound is administered in a controlled-release system. In one embodiment, a pump can be used (see Langer, supra; Sefton, CRC Crit. Ref. Biomed. Eng. 14:201 (1987); Buchwald et al., Surgery 88:507 (1980); Saudek et al., N. Engl. J Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled-release system can be placed in proximity of the target of the Benzopyranone Compounds, e.g., the brain, thus requiring only a fraction of the systemic dose (see, e.g., Goodson, in Medical Applications of Controlled Release, supra, vol. 2, pp. 115-138 (1984)). Other controlled-release systems discussed in the review by Langer (Science 249:1527-1533 (1990)) can be used.

The present compositions comprise an effective amount of a Benzopyranone Compound, in one embodiment in isolated form, together with a suitable amount of a pharmaceutically acceptable carrier so as to provide the form for proper administration to the patient.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which a Benzopyranone Compound is administered. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. In addition, auxiliary, stabilizing, thickening, lubricating and coloring agents can be used. When administered to a patient, the Benzopyranone Compounds and pharmaceutically acceptable carriers can be sterile. Water is a useful carrier when a Benzopyranone Compound is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable carrier is a capsule (see e.g., U.S. Pat. No. 5,698,155). Other examples of suitable pharmaceutical carriers are described in “Remington's Pharmaceutical Sciences” (20^{th} ed., 2000) by E. W. Martin.

In one embodiment, the Benzopyranone Compounds are formulated in accordance with routine procedures as a pharmaceutical composition adapted for intravenous administration to human beings. Typically, Benzopyranone Compounds for intravenous administration are solutions in sterile isotonic aqueous buffer. Where necessary, the compositions can also include a solubilizing agent. Compositions for intravenous administration can optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the Benzopyranone Compound is to be administered by infusion, it can be dispensed, for example, with an infusion bottle containing sterile pharmaceutical grade water or saline. Where the Benzopyranone Compound is administered by injection, an ampoule of sterile water for injection or saline can be provided so that the ingredients can be mixed prior to administration. Compositions for oral delivery can be in the form of tablets, lozenges, aqueous or oily suspensions, granules, powders, emulsions, capsules, syrups, or elixirs, for example. Orally administered compositions can contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered Benzopyranone Compounds. In these later platforms, fluid from the environment surrounding the capsule is imbibed by the driving compound, which swells to displace the agent or agent composition through an aperture. These delivery platforms can provide an essentially zero-order delivery profile as opposed to the spiked profiles of immediate release formulations. A time delay material such as glycerol monostearate or glycerol stearate can also be used. Oral compositions can include standard carriers such as mannitol, lactose, starch, magnesium stearate, sodium saccharine, cellulose or magnesium carbonate. Such carriers can be of pharmaceutical grade.

The amount of the Benzopyranone Compound that is effective for treating or preventing cancer can be determined using standard techniques. In addition, in vitro or in vivo assays can optionally be employed to help identify optimal dosage ranges. An effective dose amount can also depend on the route of administration, and the seriousness of the disease or disorder, and should be decided according to the judgment of the practitioner and each patient's circumstances. However, the general range of effective oral administration amounts of the Benzopyranone Compound is from about 0.5 mg/day to about 5000 mg/day, in one embodiment about 500 mg/day to about 3500 mg/day, in another one embodiment about 1000 mg/day to about 3000 mg/day, in another one embodiment about 1500 mg/day to about 2500 mg/day and in another one embodiment about 2000 mg/day. In another embodiment, effective amounts for intravenous administration are about 10% of an oral dosage amount and effective amounts for convection-enhanced drug administration are about 1% of an oral dosage amount. Of course, it is often practical to administer the daily dose of compound in portions, at various hours of the day. However, in any given case, the amount of Benzopyranone Compound administered will depend on such factors as the solubility of the active component, the formulation used and the route of administration. Suppositories generally contain an effective amount of a Benzopyranone Compound in the range of about 0.5% to about 10% by weight. Oral compositions can contain about 10% to about 95% of Benzopyranone Compound. In some embodiments of the invention, suitable effective dose amounts for oral administration are generally about 10-500 mg of Benzopyranone Compound per kilogram body weight. In other embodiments, the oral effective dose amount is about 10-100 mg, 100-300 mg, 300-900 mg, or 900-1500 mg per kilogram body weight. In other embodiments, the effective oral dose amount is about 100-200 mg, 200-300 mg, 300-400 mg or 400-500 mg per kilogram body weight. In other embodiments of the invention, effective dose amounts for oral administration are generally 1-7500 micrograms of Benzopyranone Compound per kilogram body weight. In other embodiments, the effective oral dose amount is about 1-10 micrograms, 10-30 micrograms, 30-90 micrograms, or 90-150 micrograms per kilogram body weight. In other embodiments, the effective oral dose amount is about 150-250 micrograms, 250-325 micrograms, 325-450 micrograms, 450-1000 micrograms or 1000-7500 micrograms per kilogram body weight. Effective dose amounts can be extrapolated from dose-response curves derived from in vitro or animal model test systems. Such animal models and systems are well known in the art.

The invention also provides pharmaceutical packs or kits comprising one or more containers containing one or more Benzopyranone Compounds. Optionally associated with such container(s) can be a notice in the form prescribed by a governmental agency regulating the manufacture, use or sale of pharmaceuticals or biological products, which notice reflects approval by the agency of manufacture, use or sale for human administration. In certain embodiments, the kit can also contain one or more other chemotherapeutic agents that can be administered prior to, subsequent to or concurrently with a Benzopyranone Compound.

The Benzopyranone Compounds can be assayed in vitro, and then in vivo, for the desired therapeutic or prophylactic activity, prior to use in humans. For example, in vitro assays can be used to determine whether administration of a specific Benzopyranone Compound or combination of Benzopyranone Compounds is preferred.

In one embodiment, a patient tissue sample is grown in culture and contacted with or otherwise administered a Benzopyranone Compound, and the effect of the Benzopyranone Compound upon the tissue sample is observed and compared with a non-contacted tissue. In other embodiments, a cell culture model is used in which the cells of the cell culture are contacted with or otherwise administered a Benzopyranone Compound, and the effect of such Benzopyranone Compound upon the tissue sample is observed and compared with a non-contacted cell culture. Generally, a lower level of proliferation or survival of the contacted cells compared to the non-contracted cells indicates that the Benzopyranone Compound is effective to treat a patient having cancer. Such Benzopyranone Compounds can also be demonstrated effective and safe using animal model systems.

The Benzopyranone Compounds can be in the form of a pharmaceutically acceptable salt. Pharmaceutically acceptable are conveniently formed, as is usual in organic chemistry, by reacting a free-base form of a Benzopyranone Compound with a suitable acid, such as have been described above. The salts can be formed in high yields at moderate temperatures, and can be prepared by isolating the salt form of a Benzopyranone Compound from a suitable acidic wash in the final step of a synthesis. The salt-forming acid can be dissolved in an anhydrous or a water-containing organic solvent, such as an alkanol, such as methanol, ethanol or isopropanol; ketone, such as acetone; or ester, such as ethyl acetate. On the other hand, if a free-base form of a Benzopyranone Compound is desired, it can be isolated from a basic final wash step. A typical technique for preparing hydrochloride salts is to dissolve the free base in a suitable solvent and dry the solution thoroughly, as over molecular sieves, before bubbling hydrogen chloride gas through it.

### 4.6 Additional Therapies

The methods for treating or preventing cancer comprising the administration of an effective amount of a Benzopyranone Compound can further comprise the adminstration of an effective amount of other therapy. The other therapy includes, but is not limited to, chemotherapy, radiation therapy, hormonal therapy, a bone marrow transplant, stem-cell replacement therapy, another biological therapy and an immunotherapy.

In one embodiment, the methods of the invention further comprise the administration of an angiogenesis inhibitor such as but not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); Fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; an antiinflammatory steroid such as but not limited to dexamethasone; a Metalloproteinase inhibitor (TIMP); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; a Retinoids Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; ZD 6474; a farnesyl transferase inhibitor (FTI); and a bisphosphonate (e.g., alendronate, etidronate, pamidronate, risedronate, ibandronate, zoledronate, olpadronate, icandronate or neridronate).

The other therapy can be the administration of an anti-cancer agent. Useful anti-cancer agents include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; erbitux; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; ImiDs™; interleukin II (including recombinant interleukin II, or rIL2), interferon-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; SelCID®; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment the anti-cancer agent is 5-fluorouracil or leucovorin, which can also be administered prior to, subsequent to or concurrently with the administration of an effective amount of thalidomide or a topoisomerase inhibitor.

The other therapy can be radiation therapy, comprising the use of x-rays, gamma rays and other sources of radiation to destroy the cancer cells. In some embodiments, the radiation treatment is administered as external beam radiation or teletherapy wherein the radiation is directed from a remote source. In other embodiments, the radiation treatment is administered as internal therapy or brachytherapy wherein a radioactive source is placed inside the body close to cancer cells or a tumor mass.

### 4.7 Inhibition of Cancer and Neoplastic Cells and Disease

The Benzopyranone Compounds can be demonstrated to inhibit tumor cell proliferation, cell transformation and/or tumorigenesis in vitro and in vivo using a variety of assays known in the art, or described herein. Such activity can be demonstrated in an in vitro assay by contacting a Benzopyranone Compound with a tumor cell. In general, a tumor cell is exposed to varying concentrations of a Benzopyranone Compound, followed by measuring cell survival relative to a control. Such assays can use cells of a cancer cell line, or cells from a patient. Many assays well-known in the art can be used to assess such survival and/or growth; for example, cell proliferation can be assayed by measuring (³H)-thymidine incorporation, by direct cell count, by detecting changes in transcription, translation or activity of known genes such as proto-oncogenes (e.g.,fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as Western blotting or immunoprecipitation using commercially available antibodies (for example, many cell-cycle marker antibodies are from Santa Cruz Inc.). mRNA can be quantitated by methods that are well known and routine in the art, for example, using northern analysis, RNase protection or the polymerase chain reaction in connection with the reverse transcription. Cell viability can be assessed by using trypan-blue staining or other cell death or viability markers known in the art. Differentiation can be assessed visually based on changes in morphology, for example.

The Benzopyranone Compounds can also be demonstrated to inhibit glioma tumor cell proliferation, cell transformation and tumorigenesis in vitro and in vivo using a variety of assays known in the art, or described herein. Such activity can be demonstrated in an in vitro assay by contacting a Benzopyranone Compound with a glioma tumor cell. (Haroun, R. I. et al., J. Neurooncol. 58:115-23 (2002); Sharma A. et al., J. Mol. Neurosci. 17:331-9 (2001); Iwadate Y. et al., Int. J. Mol. Med. 10:187-92 (2002)).

The present invention provides for cell-cycle and cell-proliferation analysis by a variety of techniques known in the art, including but not limited to the following examples.

As one example, bromodeoxyuridine (BRDU) incorporation can be used as an assay to identify proliferating cells. The BRDU assay identifies a cell population undergoing DNA synthesis by incorporation of BRDU into newly synthesized DNA. Newly synthesized DNA can then be detected using an anti-BRDU antibody (see Hoshino et al., Int. J Cancer 38:369 (1986); Campana et al., J. Immunol. Meth. 107:79 (1988)).

Cell proliferation can also be examined using (³H)-thymidine incorporation (see e.g., Chen, J., Oncogene 13:1395-403 (1996); Jeoung, J., J. Biol. Chem. 270:18367-73 (1995)). This assay allows for quantitative characterization of S-phase DNA synthesis. In this assay, cells synthesizing DNA will incorporate (³H)-thymidine into newly synthesized DNA. Incorporation can then be measured by standard techniques in the art such as by counting of radioisotope in a Scintillation counter (e.g. Beckman LS 3800 Liquid Scintillation Counter).

Detection of proliferating cell nuclear antigen (PCNA) can also be used to measure cell proliferation. PCNA is a 36 kilodalton protein whose expression is elevated in proliferating cells, particularly in early G1 and S phases of the cell cycle and therefore can serve as a marker for proliferating cells. Positive cells are identified by immunostaining using an anti-PCNA antibody (see Li et al., Curr. Biol. 6:189-199 (1996); Vassilev et al., J. Cell Sci. 108:1205-15 (1995)).

Cell proliferation can be measured by counting samples of a cell population over time (e.g. daily cell counts). Cells can be counted using a hemacytometer and light microscopy (e.g. HyLite hemacytometer, Hausser Scientific). Cell number can be plotted against time in order to obtain a growth curve for the population of interest. In a preferred embodiment, cells counted by this method are first mixed with the dye Trypan-blue (Sigma), such that living cells exclude the dye, and are counted as viable members of the population.

DNA content and/or mitotic index of the cells can be measured, for example, based on the DNA ploidy value of the cell. For example, cells in the G1 phase of the cell cycle generally contain a 2N DNA ploidy value. Cells in which DNA has been replicated but have not progressed through mitosis (e.g. cells in S-phase) will exhibit a ploidy value higher than 2N and up to 4N DNA content. Ploidy value and cell-cycle kinetics can be further measured using propidum iodide assay (see e.g. Turner, T., et al., Prostate 34:175-81 (1998)). Alternatively, the DNA ploidy can be determined by quantitation of DNA Feulgen staining (which binds to DNA in a stoichiometric manner) on a computerized microdensitometrystaining system (see e.g., Bacus, S., Am. J. Pathol. 135:783-92 (1989)). In an another embodiment, DNA content can be analyzed by preparation of a chromosomal spread (Zabalou, S., Hereditas 120:127-40 (1994); Pardue, Meth. Cell Biol. 44:333-351 (1994)).

The expression of cell-cycle proteins (e.g., CycA. CycB, CycE, CycD, cdc2, Cdk4/6, Rb, p21, p27, etc.) provide crucial information relating to the proliferative state of a cell or population of cells. For example, identification in an anti-proliferation signaling pathway can be indicated by the induction of p21^{cip1}. Increased levels of p21 expression in cells results in delayed entry into G1 of the cell cycle (Harper et al., Cell 75:805-816 (1993); Li et al., Curr. Biol. 6:189-199 (1996)). p21 induction can be identified by immunostaining using a specific anti-p21 antibody available commercially (e.g. Santa Cruz). Similarly, cell-cycle proteins can be examined by Western blot analysis using commercially available antibodies. In another embodiment, cell populations are synchronized prior to detection of a cell cycle protein. Cell cycle proteins can also be detected by FACS (fluorescence-activated cell sorter) analysis using antibodies against the protein of interest.

Detection of changes in length of the cell-cycle or speed of cell-cycle can also be used to measure inhibition of cell proliferation by the Benzopyranone Compounds. In one embodiment the length of the cell-cycle is determined by the doubling time of a population of cells (e.g., using cells contacted or not contacted with one or more Benzopyranone Compounds of the invention). In another embodiment, FACS analysis is used to analyze the phase of cell-cycle progression, or purify G1, S, and G2/M fractions (see e.g., Delia, D. et al., Oncogene 14:2137-47 (1997)).

Lapse of cell-cycle checkpoint(s), and/or induction of cell-cycle checkpoint(s), can be examined by the methods described herein, or by any method known in the art. Without limitation, a cell-cycle checkpoint is a mechanism which ensures that a certain cellular events occur in a particular order. Checkpoint genes are defined by mutations that allow late events to occur without prior completion of an early event (Weinert, T., and Hartwell, L., Genetics, 134:63-80 (1993)). Induction or inhibition of cell-cycle checkpoint genes can be assayed, for example, by Western blot analysis, or by immunostaining, etc. Lapse of cell-cycle checkpoints can be further assessed by the progression of a cell through the checkpoint without prior occurrence of specific events (e.g., progression into mitosis without complete replication of the genomic DNA).

In addition to the effects of expression of a particular cell-cycle protein, activity and post-translational modifications of proteins involved in the cell-cycle can play an integral role in the regulation and proliferative state of a cell. The invention provides for assays involved detected post-translational modifications (e.g. phosphorylation) by any method known in the art. For example, antibodies that detect phosphorylated tyrosine residues are commercially available, and can be used in Western blot analysis to detect proteins with such modifications. In another example, modifications such as myristylation, can be detected on thin layer chromatography or reverse phase h.p.l.c. (see e.g., Glover, C., Biochem. J. 250:485-91 (1988); Paige, L., Biochem J. 250:485-91 (1988)).

Activity of signaling and cell cycle proteins and/or protein complexes is often mediated by a kinase activity. The present invention provides for analysis of kinase activity by assays such as the histone H1 assay (see e.g., Delia, D. et al., Oncogene 14:2137-47 (1997)).

The Benzopyranone Compounds can also be demonstrated to alter cell- proliferation in cultured cells in vitro using methods which are well known in the art. Specific examples of cell-culture models for primary brain cancer and brain metastasis include, but are not limited to, those found in the following U.S. Pat. Nos. 6,194,158; 6,051,376 and 6,071,696.

The Benzopyranone Compounds can also be demonstrated to inhibit cell transformation (or progression to malignant phenotype) in vitro. In this embodiment, cells with a transformed cell phenotype are contacted with one or more Benzopyranone Compounds, and examined for change in characteristics associated with a transformed phenotype (a set of in vitro characteristics associated with a tumorigenic ability in vivo), for example, but not limited to, colony formation in soft agar, a more rounded cell morphology, looser substratum attachment, loss of contact inhibition, loss of anchorage dependence, release of proteases such as plasminogen activator, increased sugar transport, decreased serum requirement, or expression of fetal antigens, etc. (see Luria et al., 1978, General Virology, 3d Ed., John Wiley & Sons, New York, pp. 436-446).

Loss of invasiveness or decreased adhesion can also be used to demonstrate the anti-cancer effects of the Benzopyranone Compounds. For example, a critical aspect of the formation of a metastatic cancer is the ability of a precancerous or cancerous cell to detach from primary site of disease and establish a novel colony of growth at a secondary site. The ability of a cell to invade peripheral sites is reflective of a potential for a cancerous state. Loss of invasiveness can be measured by a variety of techniques known in the art including, for example, induction of E-cadherin-mediated cell-cell adhesion. Such E-cadherin-mediated adhesion can result in phenotypic reversion and loss of invasiveness (Hordijk et al., Science 278:1464-66 (1997)).

Loss of invasiveness can be further examined by inhibition of cell migration. A variety of 2-dimensional and 3-dimensional cellular matrices are commercially available (Calbiochem-Novabiochem Corp. San Diego, Calif.). Cell migration across or into a matrix can be examined by microscopy, time-lapsed photography or videography, or by any method in the art allowing measurement of cellular migration. In a related embodiment, loss of invasiveness is examined by response to hepatocyte growth factor (HGF). HGF-induced cell scattering is correlated with invasiveness of cells such as Madin-Darby canine kidney (MDCK) cells. This assay identifies a cell population that has lost cell scattering activity in response to HGF (Hordijk et al., Science 278:1464-66 (1997)).

Alternatively, loss of invasiveness can be measured by cell migration through a chemotaxis chamber (Neuroprobe/Precision Biochemicals Inc. Vancouver, BC). In such assay, a chemo-attractant agent is incubated on one side of the chamber (e.g., the bottom chamber) and cells are plated on a filter separating the opposite side (e.g., the top chamber). In order for cells to pass from the top chamber to the bottom chamber, the cells must actively migrate through small pores in the filter. Checkerboard analysis of the number of cells that have migrated can then be correlated with invasiveness (see e.g., Ohnishi, T., Biochem. Biophys. Res. Commun. 193:518-25 (1993)).

The Benzopyranone Compounds can also be demonstrated to inhibit tumor formation in vivo. A vast number of animal models of hyperproliferative disorders, including tumorigenesis and metastatic spread, are known in the art (see Table 317-1, Chapter 317, “Principals of Neoplasia,” in Harrison's Principals of Internal Medicine, 13th Edition, Isselbacher et al., eds., McGraw-Hill, New York, p. 1814, and Lovejoy et al., 1997, J. Pathol. 181:130-135). Specific examples for primary brain cancer and brain metastasis can be found in the following U.S. Pat. Nos. 5,894,018; 6,028,174 and 6,203,787, which are incorporated by reference herein. Further, general animal models applicable to many types of cancer have been described, including, but not restricted to, the p53-deficient mouse model (Donehower, 1996, Semin. Cancer Biol. 7:269-278), the Min mouse (Shoemaker et al., Biochem. Biophys. Acta, 1332:F25-F48 (1997)), and immune responses to tumors in rat (Frey, Methods, 12:173-188 (1997)).

For example, a Benzopyranone Compound can be administered to a test animal, preferably a test animal predisposed to develop a tumor, and the test animal subsequently examined for an decreased incidence of tumor formation in comparison with controls not administered the Benzopyranone Compound. Alternatively, a Benzopyranone Compound can be administered to test animals having a tumor (e.g., animals in which a tumor has been induced by introduction of malignant, neoplastic, or transformed cells, or by administration of a carcinogen) and subsequently examining the tumor in the test animals for tumor regression in comparison to control animals not administered the Benzopyranone Compound.

The following illustrative examples are set forth to assist in understanding the invention and do not limit the invention described and claimed herein.

The following examples are non-limiting aspects of the invention.

### 5. EXAMPLES

Example 1 relates to the synthesis of Compound 1, an illustrative Benzopyranone Compound, set forth in Table 1. Compound 1 was prepared according to the scheme set forth below.

### 5.1 Example 1

Synthesis of Compound 1

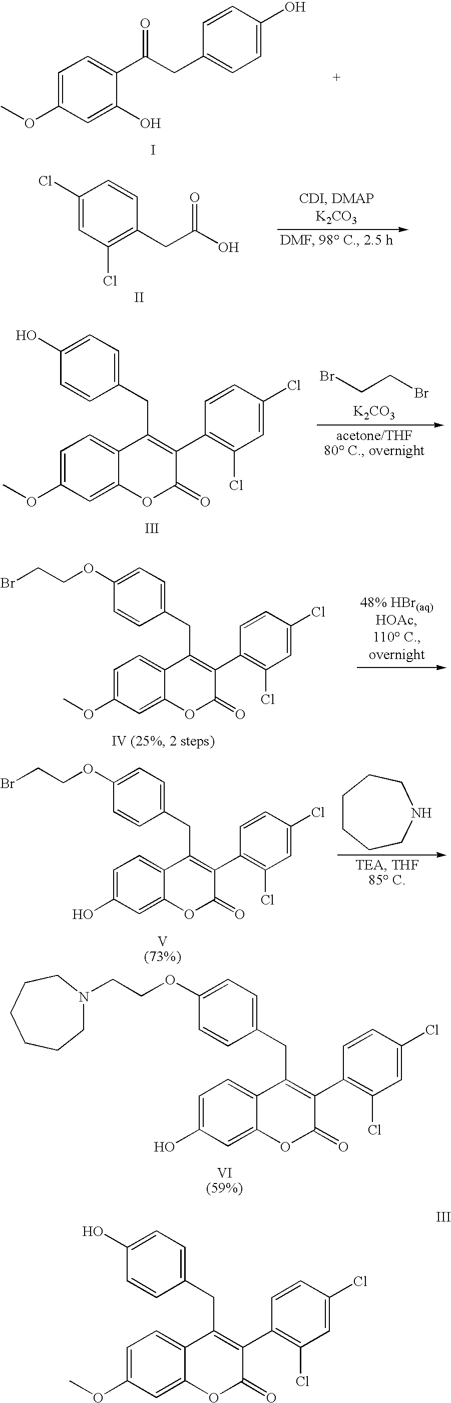

Compound III. 2,4-Dichlorophenyl acetic acid (30.0 g, 147.3 mmol, 1.0 equiv., purchased from Aldrich), ketone I (60.4 g, 294.6 mmol, 2.0 equiv.), potassium carbonate (101.8 g, 736.5 mmol, 5.0 equiv.) and DMAP (9.0 g, 73.7 mmol, 0.5 equiv.) were placed in a 1.0 L round-bottomed flask equipped with a stir bar. The solids were suspended in anhydrous DMF (300 mL) and cooled in an ice bath with stirring. 1,1′-Carbonyl-diimidazole (CDI) (59.7 g, 368.3 mmol, 2.5 equiv., purchased from Aldrich) was added in small portions through a powder funnel over about a 15 minute period. The mixture was allowed to warm to room temperature and stirred until gas evolution had ceased. The reaction flask was equipped with a reflux condenser and heated to about 98° C. for about 2.5 hours. The reaction was monitored using LCMS. After the solvent was removed in vacuo, water (1.0 L) was added to the crude product, and the precipitate collected using vacuum filtration. The product was dried under high vacuum at about 50° C. overnight to give compound III as a yellow solid (44.8 g).

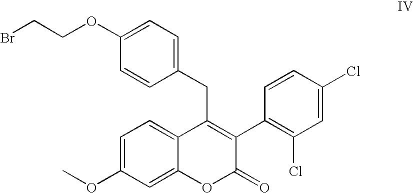

Compound IV. Crude compound III (44.8 g) and potassium carbonate (87.0 g, 629 mmol, 6.0 equiv) were placed in a 1.0 L round-bottomed flask equipped with a stir bar and condenser. The solids were suspended in acetone (400 mL) and THF (200 mL). 1,2-dibromoethane (54.0 mL, 629 mmol, 6.0 equiv.) was added using a syringe and the reaction mixture heated to about 80° C. for about 17 h. The reaction was monitored using LCMS. Upon completion, the reaction mixture was filtered and the solids washed with acetone (˜200 mL), the filtrate was concentrated in vacuo and adsorbed onto silica gel. Flash chromatography (22:78 EtOAc:Hex) afforded compound IV as a white solid (13.83 g, 25.9 mmol, 25% yield over 2 steps). LCMS (m/z) M+1=533.2.

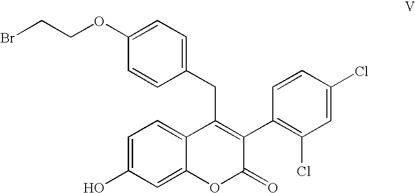

Compound V. Alkyl bromide V (8.63 g, 16.2 mmol, 1.0 equiv.) was placed in a 500 mL round-bottomed flask equipped with a stir bar and condenser and then dissolved in acetic acid (150 mL) and 48% aqueous HBr (150 mL). The reaction mixture was heated to about 110° C. and stirred overnight. The reaction was monitored using LCMS. Upon completion of the reaction, acetic acid was removed in vacuo, EtOAc (500 mL) was added to the crude product and washed with saturated NaHCO₃ (300 mL). The aqueous layer was extracted with EtOAc (2×200 mL). The combined organic layers were washed with brine (500 mL), dried with MgSO₄, and then concentrated in vacuo onto silica gel. Flash chromatography (40:60 EtOAc:Hex) afforded compound V as a light yellow solid (6.15 g, 11.8 mmol, 73% yield). LCMS (m/z) M+1=519.3.

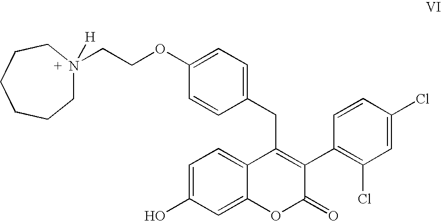

Compound VI. In a 500 mL round-bottomed flask equipped with a stir bar and condenser was added intermediate V (4.97 g, 9.55 mmol, 1.0 equiv.). Anhydrous THF (200 mL), hexamethyleneimine (3.3 mL, 28.7 mmol, 3.0 equiv.), and triethylamine (5.3 mL, 38.2 mmol, 4.0 equiv.) were added using a syringe, and the reaction mixture was heated to about 85° C. for 16 h. Volatiles were removed in vacuo and the crude product was subjected to high vacuum for about 1 h. The crude material was dissolved in MeOH (60 mL) and purified using preparative liquid chromatography (20-100% H₂O/MeCN, over 13 runs). Product containing fractions were combined and volatiles were removed in vacuo. The resulting material was dissolved in EtOAc (800 mL) and washed with saturated NaHCO₃ (800 mL). The layers were separated and the aqueous layer was extracted with EtOAc (2×200 mL). The organic layers were combined, dried with MgSO₄, and concentrated in vacuo to give a light yellow solid (3.02 g, 5.61 mmol, 59%) of the free amine. The purified product was dissolved in anhydrous dichloromethane (20 mL), 2.0N HCl in diethyl ether (4.0 mL) was added and the mixture was concentrated in vacuo. 1H NMR (d6-DMSO, 400 MHz) δ 10.73 (s, 1H), 10.42 (bs, 1H), 7.74 (d, J=2.0 Hz, 1H), 7.44-7.50 (m, 3H), 7.05 (d, J =8.0 Hz, 2H), 6.85 (d, J=6.8 Hz, 2H), 6.80 (d, J=2.4 Hz, 1H), 6.75 (dd, J=2.4, 8.8 Hz, 1H), 4.31 (m, 2H), 4.03 (d, J=15.1 Hz, 1H), 3.74 (d, J=15.1 Hz, 1H), 3.31-3.48 (m, 4H), 3.15-3.22 (m, 2H), 1.80-1.83 (m, 4H), 1.54-1.66 (m, 4H); LCMS (m/z) M+1=538.3; Anal. Calcd for C₃₀H₃₀NO₄Cl₃: C, 62.67; H, 5.26; N, 2.44. Found C, 62.54; H, 5.22; N, 2.25.

### 5.2 Example 2

Cell Proliferation Assay

Cells are plated at validated densities for each cell type in 96-well plates (plating densities were validated to be in the linear range for the course of the assay). The following day, the cells are treated with varying concentrations of a Benzopyranone Compound in 0.2% DMSO and the plates are incubated for 3 days at 37° C. with 5% CO₂. Following the 3 day treatment, proliferation/viability is assessed by MTT assay (adherent cell lines) or Alamar Blue (suspension cell lines). Absorbance/fluorescence is then measured and the percent of DMSO values are calculated for each Benzopyranone Compound concentration and IC₅₀ values are calculated using ActivityBase. Each concentration is tested in triplicate.

### 5.3 Example 3

Apoptosis Assay

Apoptosis is evaluated using the Homogeneous Caspase Assay (Roche). Cells are plated and then treated in triplicate the following day with 10 μM of a Benzopyranone Compound in 0.2% DMSO. Cells are incubated with the Benzopyranone Compound for 24 hours and then assessed for caspase activity. OD readings are obtained at 405 nm and all readings are averaged for triplicate treatments and compared to DMSO-treated cells.

### 5.4 Example 4

In Vivo Efficacy Studies (U87-MG, HT-29, MDA-MB-231)

Tumor cells (U87-MG, HT-29 or MDA-MB-231) (in 0.1 ml PBS) are injected subcutaneously into the hind legs of female C.B-17 SCID mice (6-8 weeks; Charles River). After 7-10 days of tumor cell inoculation, mice bearing tumors of 75-125 mm³ are pooled together and randomized into various groups. The mice are treated i.p. with Benzopyarnone Compounds suspended in CMC/Tween (0.5% carboxymethyl cellulose+0.05% Tween-80 in water). Mice are individually followed throughout the experiment and all mice are weighed twice weekly, and tumor measurements are taken by digital calipers twice weekly. Tumor measurements are calculated using the formula (W²×L)/2.

The present invention is not to be limited in scope by the specific embodiments disclosed in the examples which are intended as illustrations of a few aspects of the invention, and any embodiments which are functionally equivalent are within the scope of this invention. Indeed, various modifications of the invention in addition to those shown and described herein will become apparent to those skilled in the art and are intended to fall within the appended claims.

A number of references have been cited, the entire disclosures of which are incorporated herein by reference.

What is claimed is:

1. A compound of the formula: 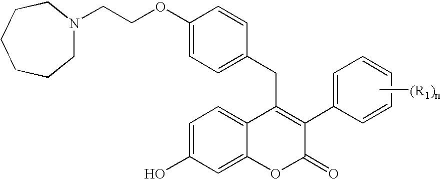
or a pharmaceutically acceptable salt thereof, wherein:
R₁ is at each occurrence independently halogen or trifluoromethyl; and
n is 1, 2or 3.

2. The compound of claim 1, wherein R₁ is halogen.

3. The compound of claim 2 wherein halogen is fluoro.

4. The compound of claim 2, wherein halogen is chloro.

5. The compound of claim 1, wherein R₁ is trifluoromethyl.

6. The compound of claim 1, wherein n is 1.

7. The compound of claim 1, wherein n is 2.

8. The compound of claim 1, wherein n is 3.

9. A compound having the formula: 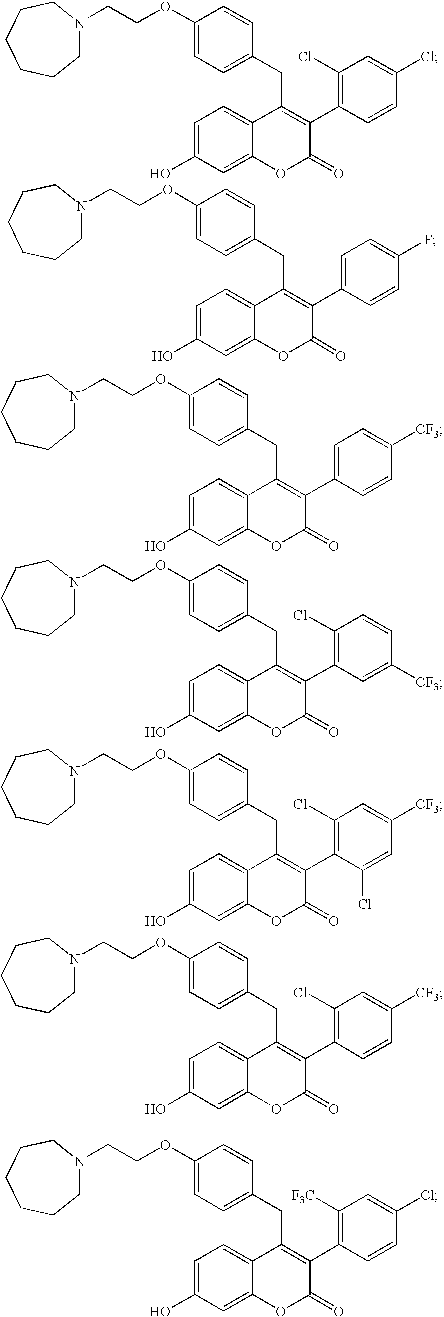
or a pharmaceutically acceptable salt thereof.

10. A pharmaceutical composition comprising a compound of claim 1, or a pharmaceutically acceptable salt thereof, and a pharmaceutically acceptable carrier or vehicle.

11. A pharmaceutical composition comprising a compound of claim 9, or a pharmaceutically acceptable salt thereof, and a pharmaceutically acceptable carrier or vehicle.

12. A single unit dosage form comprising a compound of claim 1, and a pharmaceutically acceptable carrier, excipient or diluent.

13. The single unit dosage form of claim 12, suitable for oral or mucosal administration.

14. The single unit dosage form of claim 12, suitable for parenteral administration.

15. A single unit dosage form comprising a compound of claim 9, and a pharmaceutically acceptable carrier, excipient or diluent.

16. The single unit dosage form of claim 15, suitable for oral or mucosal administration.

17. The single unit dosage form of claim 15, suitable for parenteral administration.

18. A method for treating or preventing cancer in a patient, comprising administering to a patient in need thereof an effective amount of a compound of the formula: 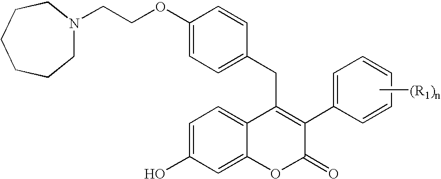
or a pharmaceutically acceptable salt thereof, wherein:
R₁ is at each occurrence independently halogen or trifluoromethyl; and
n is 1, 2 or 3.

19. The method of claim 18, wherein R₁ is halogen.

20. The method of claim 19, wherein halogen is fluoro.

21. The method of claim 19, wherein halogen is chloro.

22. The method of claim 18, wherein R₁ is trifluoromethyl.

23. The method of claim 18, wherein n is 1.

24. The method of claim 18, wherein n is 2.

25. The method of claim 18, wherein n is 3.

26. The method of claim 18, wherein the cancer is a primary brain cancer.

27. The method of claim 18, wherein the cancer is of the head, neck, eye, mouth, throat, esophagus, chest, bone, lung, colon, rectum, stomach, prostate, breast, ovary, testicle or other reproductive organ, skin, thyroid, blood, lymph node, kidney, liver, pancreas, brain or central nervous system.

28. The method of claim 18, wherein the cancer is a primary intracranial central nervous system tumor.

29. The method of claim 28, wherein the primary intracranial central nervous system tumor is glioblastoma multiforme; malignant astrocytoma; oligdendroglioma; ependymoma; a low-grade astrocytoma; meningioma; mesenchymal tumor; pituitary tumor; nerve sheath tumor such as a schwannoma; central nervous system lymphoma; medulloblastoma; primitive neuroectodermal tumor; neuron and neuron/glial tumor; craniopharyngioma; germ cell tumor; or choroid plexus tumor.

30. The method of claim 18, wherein the cancer is a primary spinal tumor.

31. The method of claim 30 wherein the primary spinal tumor is a schwannoma, a meningioma, an ependymoma, a sarcoma, an astrocytoma, a glioma, a vascular tumor, a chordoma or an epidermoid.

32. The method of claim 18, wherein the cancer has metastasized.

33. The method of claim 32, wherein the metastasized cancer originated in the lung (both small cell or non-small cell), breast, from an unknown primary tumor, a melanoma or colon.

34. The method of claim 18, wherein the cancer is a solid tumor.

35. The method of claim 34, wherein the solid tumor is of the breast, colon, prostate, pancreas, ovaries or uterus.

36. The method of claim 34, wherein the solid tumor is a glioma or non-small cell lung cancer.

37. The method of claim 18, wherein the cancer is leukemia.

38. A method for inhibiting the growth of a cancer cell or neoplastic cell comprising contacting a cancer cell or neoplastic cell with an effective amount of a compound of the formula: 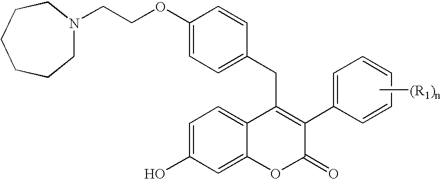
or a pharmaceutically acceptable salt thereof, wherein:
R₁ is at each occurrence independently halogen or trifluoromethyl; and
n is 1, 2 or 3.

39. The method of claim 38, wherein R₁ is halogen.

40. The method of claim 39, wherein halogen is fluoro.

41. The method of claim 39, wherein halogen is chloro.

42. The method of claim 38, wherein R₁ is trifluoromethyl.

43. The method of claim 38, wherein n is 1.

44. The method of claim 38, wherein n is 2.

45. The method of claim 38, wherein n is 3.
